(19) United States
(12) Patent Application Publication
(10) Pub. No.: US 2025/0041666 A1
(43) Pub. Date: Feb. 6, 2025

(54) JUMP COUNTING METHOD, ELECTRONIC DEVICE AND STORAGE MEDIUM

(71) Applicants: DARIN CHEN, SHENZHEN (CN); YANG LI, SHENZHEN (CN)

(72) Inventors: DARIN CHEN, SHENZHEN (CN); YANG LI, SHENZHEN (CN)

(21) Appl. No.: 18/922,419

(22) Filed: Oct. 22, 2024

Related U.S. Application Data
(63) Continuation of application No. PCT/CN2023/089527, filed on Apr. 20, 2023.

(51) Int. Cl. A63B 24/00; A63B 5/20; G06T 7/70; G06V 40/16; G06V 40/20

(52) U.S. Cl. CPC A63B 24/0062; A63B 5/20; G06T 7/70; G06V 40/161; G06V 40/20; G06T 2207/10016; G06T 2207/30201

(57) ABSTRACT

Provided are a jump counting method, an electronic device and a storage medium. In the method, during a jump process of a detected object, a height relationship between a maximum height of a target part of the detected object measured when the detected object jumps up and a height of a jump reference line is determined, where a height of the target part of the detected object measured when the detected object stands is lower than the height of the jump reference line. The number of jumps of the detected object is counted based on the height relationship.

### CROSS-REFERENCE OF RELATED APPLICATIONS

This application is a continuation of International Application No. PCT/CN2023/089527, filed Apr. 20, 2023, which is herein incorporated by reference in its entirety.

### TECHNICAL FIELD

The disclosure relates to the technical field of artificial intelligence, and particularly to a jump counting method, an electronic device and a storage medium.

### BACKGROUND

With the development of quality education, more and more sports are included in assessments of students. Rope skipping has become an important sport for entertainment or competition on many occasions. In the related art, manual counting is mostly adopted for the rope skipping, which is however time-consuming, laborious and easily influenced by subjective factors. Therefore, in the related art, there is a problem of low efficiency in counting the rope skipping.

### SUMMARY

In view of this, the present disclosure provides a jump counting method, an electronic device and a storage medium.

In a first aspect, embodiments of the present disclosure provide a jump counting method, and the method includes: during a jump process of a detected object, determining a height relationship between a maximum height of a target part of the detection object measured when the detected object jumps up and a height of a jump reference line, where a height of the target part of the detected object measured when the detected object stands is lower than the height of the jump reference line; and counting the number of jumps of the detected object, based on the height relationship.

In a second aspect, the embodiments of the present disclosure provide an electronic device including a memory and a processor. The memory is coupled to the processor, and the memory stores instructions. The instructions, when being executed by the processor, cause the processor to perform the above method.

In a third aspect, the embodiments of the present disclosure provide a non-transitory computer-readable storage medium storing program codes therein, and the program codes are invokable by a processor to implement the above method.

### BRIEF DESCRIPTION OF THE DRAWINGS

In order to explain technical schemes in the embodiments of the present disclosure more clearly, the drawings needed in the description of the embodiments are briefly introduced below. Obviously, the drawings in the description below are only some embodiments of the present disclosure. For those skilled in the art, other drawings may be obtained based on these drawings without creative work.

FIG. 1 schematically illustrates a flowchart of a jump counting method provided in an embodiment of the present disclosure.

FIG. 2 is a schematic diagram illustrating a maximum height of a target part of a detected object and a height of a jump reference line provided in an embodiment of the present disclosure.

FIG. 3 schematically illustrates a flowchart of a jump counting method provided in an embodiment of the present disclosure.

FIG. 4 is a schematic diagram illustrating a target part provided in an embodiment of the present disclosure.

FIG. 5 is a schematic diagram illustrating a height difference provided in an embodiment of the present disclosure.

FIG. 6 is a schematic diagram illustrating a height difference provided in an embodiment of the present disclosure.

FIG. 7 is a schematic diagram illustrating a hand posture provided in an embodiment of the present disclosure.

FIG. 8 is a schematic diagram illustrating a height relationship provided in an embodiment of the present disclosure.

FIG. 9 schematically illustrates a flowchart of a jump counting method provided in an embodiment of the present disclosure.

FIG. 10 schematically illustrates a flowchart of a jump counting method provided in an embodiment of the present disclosure.

FIG. 11 is a schematic diagram illustrating a video frame provided in an embodiment of the present disclosure.

FIG. 12 is a schematic diagram illustrating a video frame provided in an embodiment of the present disclosure.

FIG. 13 is a schematic diagram illustrating a video frame provided in an embodiment of the present disclosure.

FIG. 14 is a schematic diagram illustrating a video frame provided in an embodiment of the present disclosure.

FIG. 15 is a schematic diagram illustrating a video frame provided in an embodiment of the present disclosure.

FIG. 16 schematically illustrates a flowchart of a jump counting method provided in an embodiment of the present disclosure.

FIG. 17 is a schematic diagram illustrating a to-be-counted image provided in an embodiment of the present disclosure.

FIG. 18 schematically illustrates a flowchart of a jump counting method provided in an embodiment of the present disclosure.

FIG. 19 is a schematic diagram illustrating a jump reference line provided in an embodiment of the present disclosure.

FIG. 20 illustrates a display picture provided in an embodiment of the present disclosure.

FIG. 21 illustrates a block diagram of a jump counting apparatus provided in an embodiment of the present disclosure.

FIG. 22 illustrates a block diagram of an electronic device provided in an embodiment of the present disclosure that is used to execute the jump counting method provided in the embodiments of the present disclosure.

FIG. 23 illustrates a storage unit provided in an embodiment of the present disclosure that is used to store or carry program codes for implementing the jump counting method provided in the embodiments of the present disclosure.

### DETAILED DESCRIPTION OF PREFERRED EMBODIMENTS

In order to make those in the art better understand the schemes of the present disclosure, the technical schemes in the embodiments of the present disclosure will be described clearly and comprehensively with reference to the attached drawings.

Rope skipping is a kind of effective aerobic exercise, which has functions of burning fat, strengthening physical fitness, enhancing the coordination between hands and feet, enhancing body metabolism, and promoting physical and mental health. The rope skipping has become an important sport for entertainment or competition on many occasions.

In the related art, the rope skipping is generally counted based on manual counting, a special shaft counter for skipping rope, an infrared counter, or audio-based counting. However, the manual counting is time-consuming, laborious, and easily disturbed by external factors. The special shaft counter for skipping rope has a high cost and high loss, and there must be a physical skipping rope during the skipping. The infrared counting and audio-based counting each are easily limited by the environment. Therefore, in the related art, the number of jumps during skipping cannot be counted efficiently and accurately.

In view of this, the inventors propose, after long-term research, a jump counting method and apparatus, an electronic device and storage medium as provided in the embodiments of the present disclosure. With such schemes, during a jump process of a detected object, the number of jumps of the detected object is automatically counted based on a height relationship between a maximum height of a target part of the detected object and a jump reference line, which improves the efficiency of counting the number of jumps. The specific jump counting method is described in detail in the following embodiments.

In the jump counting method and apparatus, electronic device and storage medium provided in the embodiments of the present disclosure, during a jump process of a detected object, a height relationship between a maximum height of a target part of the detected object measured when the detected object jumps up and a height of a jump reference line is determined, where the height of the target part of the detected object measured when the detected object stands is lower than the height of the jump reference line. Based on the height relationship, the number of jumps of the detected object is counted. As such, during the jump process of the detected object, the number of jumps of the detected object is automatically counted based on the height relationship between the maximum height of the target part of the detected object and the jump reference line, which improves the efficiency of counting the number of jumps.

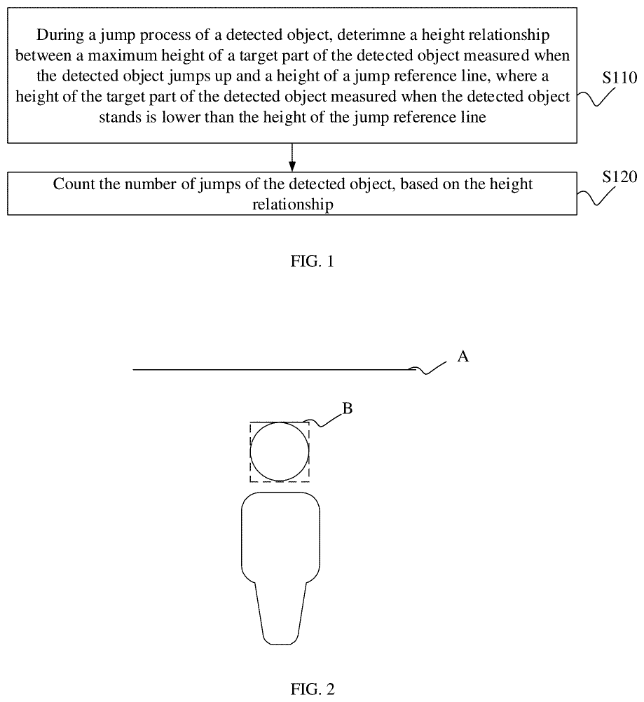

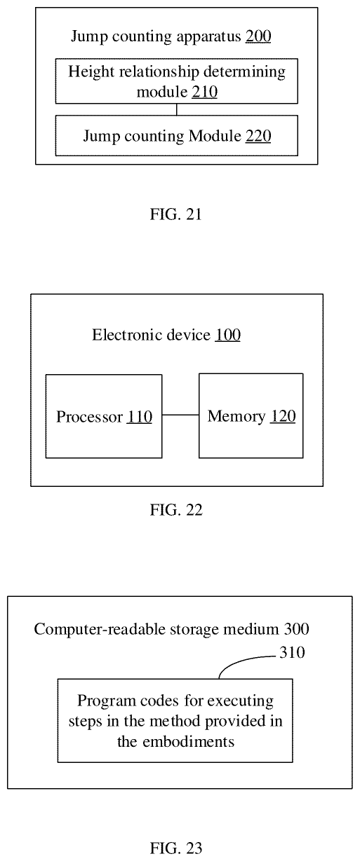

Referring to FIG. 1, a flowchart of a jump counting method provided in an embodiment of the present disclosure is schematically illustrated. With this jump counting method, during a jump process of a detected object, the number of jumps of the detected object is automatically counted based on a height relationship between a maximum height of a target part of the detected object and a jump reference line, which improves the efficiency and accuracy of counting the number of jumps. In particular, the jump counting method may be implemented by a jump counting apparatus 200 as illustrated in FIG. 21 or an electronic device 100 (as illustrated in FIG. 22) equipped with the jump counting apparatus 200. Hereinafter, the specific flow of the embodiments will be explained by taking the electronic device as an example. Of course, it is understandable that the electronic device involved in the embodiments may include a smart phone, tablet computer, robot, etc., which is not limited here. The flow illustrated in FIG. 1 will be described in detail below, and the jump counting method may specifically include the steps as follows.

In S110, during a jump process of a detected object, a height relationship between a maximum height of a target part of the detected object measured when the detected object jumps up and a height of a jump reference line is determined, where a height of the target part of the detected object measured when the detected object stands is lower than the height of the jump reference line.

In the embodiment, the electronic device may determine, during a jump process of the detected object, the height relationship between a maximum height of the target part of the detected object measured when the detected object jumps up and the height of the jump reference line, where the height of the target part of the detected object measured when the detected object stands is lower than the height of the jump reference line.

Exemplarily, referring to FIG. 2, a schematic diagram illustrating a maximum height of the target part of the detected object and the height of the jump reference line provided in an embodiment of the present disclosure is shown. As illustrated in FIG. 2, “A” represents the height of the jump reference line, and “B” represents the height of the target part measured when the detected object stands. The jump reference line may be a virtual line, and the virtual line may be visible or invisible, which is not limited here. Exemplarily, the virtual line may be drawn in an area to which the detected object faces during the jump process of the detected object, and displayed thereat so that it is visible to the detected object. The jump reference line may also be a solid line, and the jump reference line may be displayed during the jump process of the detected object.

The jump process of the detected object may be construed as a process during which the detected object jumps off the ground, and a situation that the detected object stands may be construed as a situation that the detected object does not leave the ground.

The detected object may include a human body, animal, robot and other creatures or machines that can jump. The target part of the detected object may include an upper edge of the face, eyes, nose, mouth, and the like of the detected object. There may be one or more detected objects for which the electronic device counts the number of jumps, which is not limited here.

As an implementation, the electronic device may include a camera. Further, the electronic device may collect a video stream during the jump progress of the detected object through the camera. Further, the electronic device may determine, based on the video stream, the height relationship between the maximum height of the target part of the detected object measured when the detected object jumps up and the height of the jump reference line.

As another implementation, the electronic device may include an infrared sensor. Further, the electronic device may acquire an infrared signal of the target part of the detected object during the jump progress of the detected object through the infrared sensor. Further, the electronic device may determine, based on the infrared signal, the height relationship between the maximum height of the target part of the detected object measured when the detected object jumps up and the height of the jump reference line.

As yet another implementation, the electronic device may include a radar. Further, the electronic device may acquire, during the jump process of the detected object, a radar signal indicating the height of the target part of the detected object. Further, the electronic device may determine, based on the radar signal, the height relationship between the maximum height of the target part of the detected object measured when the detected object jumps up and the height of the jump reference line.

The height relationship between the maximum height of the target part of the detected object measured when the detected object jumps up and the height of the jump reference line may include: the maximum height is equal to the height of the jump reference line, the maximum height is higher than the height of the jump reference line, or the maximum height is lower than the height of the jump reference line.

As an implementation, the electronic device may receive a first counting instruction sent by a user, and the first counting instruction may carry a first jump counting time. Accordingly, the electronic device may parse the first counting instruction to determine that there is a need to count the number of jumps, and acquire the first jump counting time. Then, within the first jump counting time, the electronic device may determine, for each jump process of the detected object, the height relationship between the maximum height of the target part of the detected object measured when the detected object jumps up and the height of the jump reference line. The first jump counting time may be 30 seconds, 1 minute, 2 minutes, etc., which is not limited here.

As another implementation, the first jump counting time is unchanged. In this case, the electronic device may store the first jump counting time in advance. When there is a need for the electronic device to count the number of jumps, the electronic device may receive a second counting instruction sent by the user, where the second counting instruction does not carry the first jump counting time. Accordingly, the electronic device may parse the second counting instruction to determine that there is a need to count the number of jumps. Then, within the first jump counting time, the electronic device may determine, for each jump process of the detected object, the height relationship between the maximum height of the target part of the detected object measured when the detected object jumps ups and the height of the jump reference line.

In some implementations, the electronic device may be in a sleep state in an initial state, and may be waked up when there is a need to count the number of jumps.

In S120, the number of jumps of the detected object is counted based on the height relationship.

In the embodiment, after the height relationship between the maximum height of the target part of the detected object measured when the detected object jumps ups and the height of the jump reference line is determined, the electronic device may count the number of jumps of the detected object based on the height relationship.

With regard to counting the number of jumps of the detected object, the electronic device may increase the number of jumps, that is, accumulate the number (i.e., increment the total number) of jumps of the detected object, or may also keep the number of jumps unchanged, that is, maintain the number of the jumps of the detected object.

In an alternative implementation, in counting the number of jumps of the detected object based on the height relationship, if it is determined, based on the height relationship, that the maximum height of the target part of the detected object measured when the detected object jumps up is higher than or equal to the height of the jump reference line, for this jump of the objected object, the electronic device may increase the number of jumps, that is, the total number of jumps of the detected object is incremented.

In an alternative implementation, in counting the number of jumps of the detected object based on the height relationship, if it is determined, based on the height relationship, that the maximum height of the target part of the detected object measured when the detected object jumps up is lower than the height of the jump reference line, for this jump of the objected object, the electronic device may keep the number of jumps unchanged, that is, the total number of jumps of the detected object is maintained. This may also be construed that, for this jump, the electronic device does not increase the number of jumps.

In some implementations, with regard to counting the number of jumps of the detected object, for each jump process of the detected object, only during a rising stage of this jump of the detected object, the electronic device may determine the height relationship between the maximum height of the target part of the detected object measured when the detected object jumps up and the height of the jump reference line, and based on the height relationship for this jump, increase or maintain the number of jumps; during a descending stage of this jump of the detected object, no determination of the height relationship is performed; and when it is determined that the detected object returns to the ground, the counting for this jump process of the detected object is finished, so as to reduce power consumption of the electronic device.

It is understandable that, in the embodiment, based on the height relationship between the maximum height of the target part of the detected object and the jump reference line, the electronic device may automatically count the number of jumps of the detected object during the jump process of the detected object, which improves the efficiency of counting the number of jumps and improves the user's experience. In addition, the way of counting, based on the height relationship, the number of jumps of the detected object (increasing the number of jumps or keeping the number of jump unchanged) improves the objectivity and accuracy of counting the number of jumps.

In the jump counting method provided in the embodiment of the present disclosure, during a jump process of the detected object, the height relationship between the maximum height of the target part of the detected object measured when the detected object jumps up and the height of the jump reference line is determined, where the height of the target part of the detected object measured when the detected object stands is lower than the height of the jump reference line. The number of jumps of the detected object is counted based on the height relationship. As such, during the jump process of the detected object, the number of jumps of the detected object is automatically counted based on the height relationship between the maximum height of the target part of the detected object and the jump reference line, which improves the efficiency of counting the number of jumps.

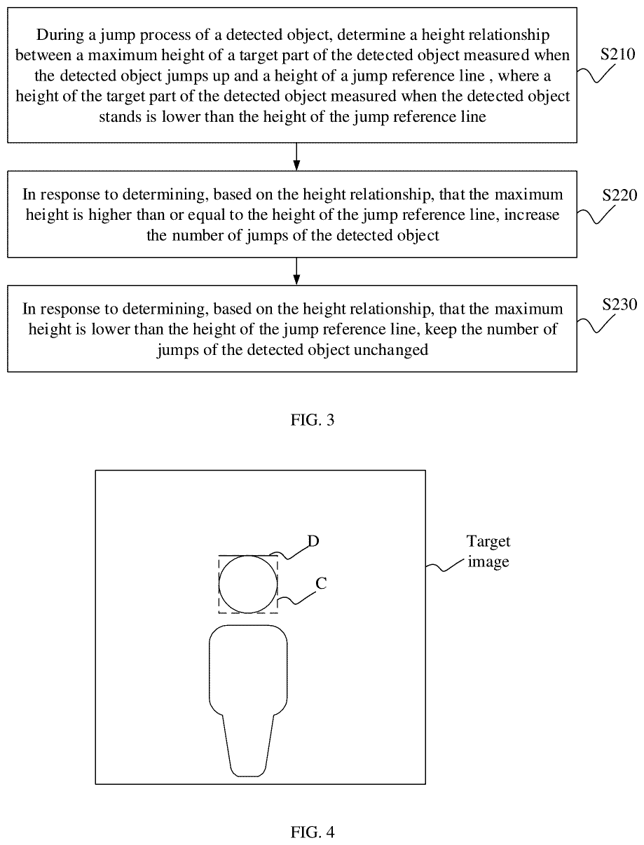

Referring to FIG. 3, a flowchart of the jump counting method provided in an embodiment of the present disclosure is schematically illustrated. The method is implemented by the electronic device mentioned above, and the flow illustrated in FIG. 3 will be described in detail below. The jump counting method may specifically include steps as follows.

In S210, during a jump process of a detected object, a height relationship between a maximum height of a target part of the detected object measured when the detected object jumps up and a height of a jump reference line is determined, where a height of the target part of the detected object measured when the detected object stands is lower than the height of the jump reference line.

In some implementations, in determining, during the jump process of the detected object, the height relationship between the maximum height of the target part of the detected object measured when the detected object jumps up and the height of the jump reference line, the electronic device may, during the jump process of the detected object, perform a face detection on the detected object to obtain a face of the detected object from the face detection, determine an upper edge of the face as the target part of the detected object, and determine the height relationship between the maximum height of the target part of the detected object measured when the detected object jumps up and the height of the jump reference line.

In an alternative implementation, the electronic device may include a camera. Further, during the jump process of the detected object, the electronic device may capture images including the detected object through the camera. The electronic device may also obtain images including the detected object captured during the jump process of the detected object, from an associated cloud or electronic device through wireless communication technology (such as Bluetooth, WiFi, zigbee and other technologies). The electronic device may also obtain images including the detected object captured during the jump process of the detected object, from an associated electronic device through a serial communication interface (such as a serial peripheral interface). Further, after obtaining the image(s) including the detected object, the electronic device may perform the face detection on the image(s) to obtain the face of the detected object.

As an implementation, in performing the face detection on the detected object to obtain the face of the detected object during the jump process of the detected object, the electronic device may, during the jump process of the detected object, capture a target image including the detected object through the camera, input the target image into a face detection model, and obtain the detected face of the detected object in the target image output by the face detection model.

The face detection model may include a Haar feature model, a Histogram of oriented gradient (HOG) feature model, a convolutional neural network (CNN), a single-step multi-frame target detection model (SSD), a multi-task convolutional neural network (MTCNN), etc., which is not limited here.

Exemplarily, after obtaining the target image including the detected object captured by the camera, the electronic device may use a cross-platform computer vision processing open source software library OpenCV together with a face detection model (such as Haarcascade feature model), to perform the face detection on the target image, to obtain the detected face of the detected object in the target image output by the face detection model. After the target image is input into Haarcascade feature model, the Haarcascade feature model may divide the target image into feature regions, where features of the image may include edge feature, linear feature, central feature and diagonal feature of the image. Further, the Haarcascade feature model may perform comparison on the divided feature regions. Exemplarily, the Haarcascade feature model may compare eyes and cheeks, lips and cheeks, and determine the face of the detected object in the target image.

In an alternative implementation, after determining the face of the detected object, the electronic device may determine the upper edge of the face as the target part of the detected object. The electronic device may also determine the eyes in the face as the target part of the detected object. The electronic device may also determine the lips in the face as the target part of the detected object, which is not limited here.

Exemplarily, referring to FIG. 4, a schematic diagram illustrating a target part provided in an embodiment of the present disclosure is shown. The electronic device may use OpenCV together with the Haarcascade feature model, to perform the face detection on the target image, and obtain a detection frame corresponding to the face of the detected object included in the target image (as indicated by “C” in FIG. 4). Further, the electronic device may determine an upper edge of the detection frame as the target part of the detected object (as indicated by “D” in FIG. 4).

As an implementation, after determining the position of the target part of the detected object in the target image, the electronic device may determine the height relationship between the maximum height of the target part of the detected object and the jump reference line in the target image. Exemplarily, the electronic device may establish a rectangular coordinate system based on the target image, and based on a relationship between a vertical coordinate of the target part of the detected object and a vertical coordinate of the jump reference line in the rectangular coordinate system, determine the height relationship between the maximum height of the target part of the detected object and the jump reference line in the target image.

In S220, in response to determining, based on the height relationship, that the maximum height is higher than or equal to the height of the jump reference line, the number of jumps of the detected object is increased.

In the embodiment, after the electronic device determines the height relationship between the maximum height of the target part of the detected object measured when the detected object jumps up and the height of the jump reference line, if it is determined, based on the height relationship, that the maximum height is higher than or equal to the height of the jump reference line, the number of jumps of the detected object may be increased. The electronic device may increase the number of jumps of the detected object based on a preset increment.

The jump reference line may be construed as a line for determining whether a jump is qualified, or a standard line for determining whether the number of jumps should be increased. When it is determined, based on the height relationship, that the maximum height of the target part of the detected object measured when the detected object jumps up is higher than or equal to the height of the jump reference line, it may be determined that this jump of the detected object is a qualified jump, and the number of jumps of the detected object is increased.

As an implementation, with regard to increasing the number of jumps of the detected object when it is determined, based on the height relationship, that the maximum height is higher than or equal to the height of the jump reference line, it may implemented as follows: when determining, based on the height relationship, that the maximum height is higher than or equal to the height of the jump reference line, the electronic device may further determine a height difference between the maximum height and the height of the jump reference line, and determine a first counting value based on the height difference. The first counting value is greater than or equal to 1. Further, the electronic device may further increase the number of jumps of the detected object based on the first counting value.

In an alternative implementation, in combination with preset relationships between height differences and first counting values, the electronic device may determine the first counting value based on the preset height difference. The preset relationships between height differences and first counting values may include a preset mapping table in which height differences are associated with first counting values in one-to-one correspondence, or a preset calculation formula of the height difference and the first counting value (for example, a result obtained by rounding a quotient of dividing the height difference by a first multiple threshold is taken as the first counting value). Alternatively, the height difference may be input into a preset model for determination of first counting value (such as a convolutional neural network, a cyclic neural network, etc.), and a result output by the preset model is obtained as the first counting value. In the embodiment, the way of determining the first counting value based on the height difference is not limited here.

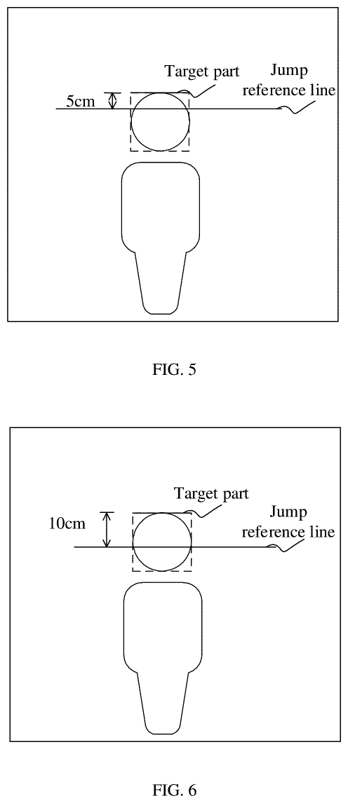

Exemplarily, relationships between height differences and first counting values may be preset in the electronic device. Exemplarily, a height difference of 5 cm corresponds to a first counting value of 1, and a height difference of 10 cm corresponds to a first counting value of 2. Exemplarily, referring to FIG. 5, a schematic diagram illustrating a height difference provided in an embodiment of the present disclosure is shown. Based on the height relationship between the maximum height of the target part of the detected object measured when the detected object jumps up and the height of the jump reference line, the electronic device may determine that the maximum height is higher than or equal to the height of the jump reference line, and determine that the height difference between the maximum height and the height of the jump reference line is 5 cm. Further, the electronic device may determine, based on the height difference, the first counting value as 1, and may increase the number of jumps of the detected object based on the first counting value, that is, the number of jumps of the detected object is incremented by 1.

Exemplarily, referring to FIG. 6, a schematic diagram illustrating a height difference provided in an embodiment of the present disclosure is shown. Based on the height relationship between the maximum height of the target part of the detected object measured when the detected object jumps up and the height of the jump reference line, the electronic device may determine that the maximum height is higher than or equal to the height of the jump reference line, and determine that the height difference between the maximum height and the height of the jump reference line is 10 cm. Further, the electronic device may determine, based on the height difference, the first counting value as 2, and may increase the number of jumps of the detected object based on the first counting value, that is, the number of jumps of the detected object is incremented by 2.

It is understandable that, the way of counting the number of jumps of the detected object may be applied to counting of the rope skipping of the detected object, and during the rope skipping of the detected object, there may be double-jump and single-jump. Based on this, in the embodiment, when determining, based on the height relationship, that the maximum height is higher than or equal to the height of the jump reference line, the electronic device may determine the height difference between the maximum height and the height of the jump reference line, determine the first counting value based on the height difference, and increase the number of jumps during the rope skipping of the detected object based on the first counting value, which improves the efficiency and accuracy of counting the skipping, and improves the user's experience.

As another implementation, with regard to increasing the number of jumps of the detected object when it is determined, based on the height relationship, that the maximum height is higher than or equal to the height of the jump reference line, it may implemented as follows: when determining, based on the height relationship, that the maximum height is higher than or equal to the height of the jump reference line, the electronic device may further determine a duration during which the maximum height is higher than or equal to the height of the jump reference line, and determine a second counting value based on the duration. The second counting value is greater than or equal to 1. Further, the electronic device may further increase the number of jumps of the detected object based on the second counting value.

In an alternative implementation, in combination with preset relationships between durations and second counting values, the electronic device may determine the second counting value based on the duration. The preset relationships between durations and second counting values may include a preset mapping table in which durations are associated with second counting values in one-to-one correspondence, or a preset calculation formula of duration and second counting value (for example, a result obtained by rounding a quotient of dividing the duration by a second multiple threshold is taken as the second counting value). Alternatively, the duration may be input into a preset model for determination of second counting value (such as a convolutional neural network, a cyclic neural network, etc.), and a result output by the preset model is obtained as the second counting value. In the embodiment, the way of determining the second counting value based on the duration is not limited here.

Exemplarily, relationships between durations and second counting values may be preset in the electronic device. Exemplarily, a duration shorter than 2 seconds corresponds to a second counting value of 1, and a duration longer than or equal to 2 seconds corresponds to a second counting value of 2.

It is understandable that, the way of counting the number of jumps of the detected object may be applied to counting the rope skipping of the detected object, and during the rope skipping of the detected object, there may be double-jump and single-jump. Based on this, in the embodiment, when determining, based on the height relationship, that the maximum height is higher than or equal to the height of the jump reference line, the electronic device may determine the duration during which the maximum height is higher than or equal to the height of the jump reference line, determine the second counting value based on the duration, and increase the number of jumps during the rope skipping of the detected object based on the second counting value, which improves the efficiency and accuracy of counting the skipping, and improves the user's experience.

As another implementation, with regard to increasing the number of jumps of the detected object when it is determined, based on the height relationship, that the maximum height is higher than or equal to the height of the jump reference line, it may implemented as follows: when determining, based on the height relationship, that the maximum height is higher than or equal to the height of the jump reference line, the electronic device may further determine a hand rotation number of the detected object, and determine a third counting value based on the hand rotation number. The third counting value is greater than or equal to 1. Further, the electronic device may further increase the number of jumps of the detected object based on the third counting value.

In an alternative implementation, in combination with preset relationships between hand rotation numbers and third counting values, the electronic device may determine the third counting value based on the hand rotation number. The preset relationships between hand rotation numbers and third counting values may include a preset mapping table in which hand rotation numbers are associated with third counting values in one-to-one correspondence, or the hand rotation number is equal to the third counting value. In the embodiment, the way of determining the third counting value based on the hand rotation number is not limited here.

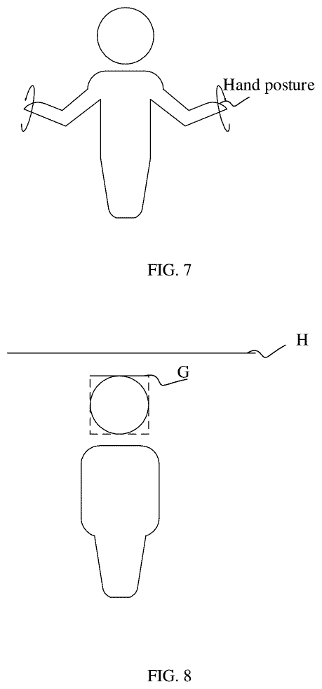

Exemplarily, referring to FIG. 7, a schematic diagram illustrating a hand posture provided in an embodiment of the present disclosure is shown. A relationship that the hand rotation number is equal to the third counting value may be preset in the electronic device. Further, if the electronic device determines, based on the height relationship, that the maximum height of the target part of the detected object measured when the detected object jumps up is higher than or equal to the height of the jump reference line, the electronic device may acquire the hand posture of the detected object during the current jump process, and determine the hand rotation number of the detected object based on the hand posture. Exemplarily, the electronic device determines the hand rotation number of the detected object as 1, and it may further determine the third counting value as 1. For another example, the electronic device determines the hand rotation number of the detected object as 2, and it may further determine the third counting value as 2.

In determining the hand rotation number of the detected object based on the hand posture, the electronic device may acquire, through the camera, a video stream of the detected object in the jump process. Further, the electronic device may determine the hand posture of the detected object based on the video stream, and determine, based on the hand posture, the hand rotation number of the detected object in the jump process.

It is understandable that, the way of counting the number of jumps of the detected object may be applied to counting the rope skipping of the detected object, and during the rope skipping of the detected object, there may be double-jump and single-jump. Based on this, in the embodiment, when determining, based on the height relationship, that the maximum height is higher than or equal to the height of the jump reference line, the electronic device may determine the hand rotation number of the detected object, determine the third counting value based on the hand rotation number, and increase the number of jumps during the rope skipping of the detected object based on the third counting value, which improves the efficiency and accuracy of counting the skipping, and improves the user's experience.

In S230, in response to determining, based on the height relationship, that the maximum height is lower than the height of the jump reference line, the number of jumps of the detected object is kept unchanged.

In some implementations, during the jump process of the detected object, if the electronic device determines, based on the height relationship between the maximum height of the detected object measured when the detected object jumps up and the height of the jump reference line, that the maximum height of the target part of the detected object is lower than the height of the jump reference line, the number of jumps of the detected object is kept unchanged.

Exemplarily, referring to FIG. 8, a schematic diagram illustrating a height relationship provided in an embodiment of the present disclosure is shown. The maximum height of the target part is indicated by “G” in FIG. 8, and the height of the jump reference line is indicated by “H” in FIG. 8. As illustrated, the maximum height is lower than the height of the jump reference line, the electronic device may determine that the current jump of the detected object is unqualified, and keep the number of jumps of the detected object unchanged.

Compared with the jump counting method illustrated in FIG. 1, the jump counting method provided in the embodiment of the present disclosure may further increase the number of jumps of the detected object if it is determined, based on the height relationship between the maximum height of the target part of the detected object measured when the detected object jumps up and the height of the jump reference line, that the maximum height is higher than or equal to the height of the jump reference line, or keep the number of jumps of the detected object unchanged if it is determined, based on the height relationship, that the maximum height is lower than the height of the jump reference line. As such, the way of counting, based on the height relationship, the jumps of the detected object (increasing the number of jumps or keeping the number of jump unchanged) improves the efficiency and accuracy of counting the number of jumps, and improves the user's experience.

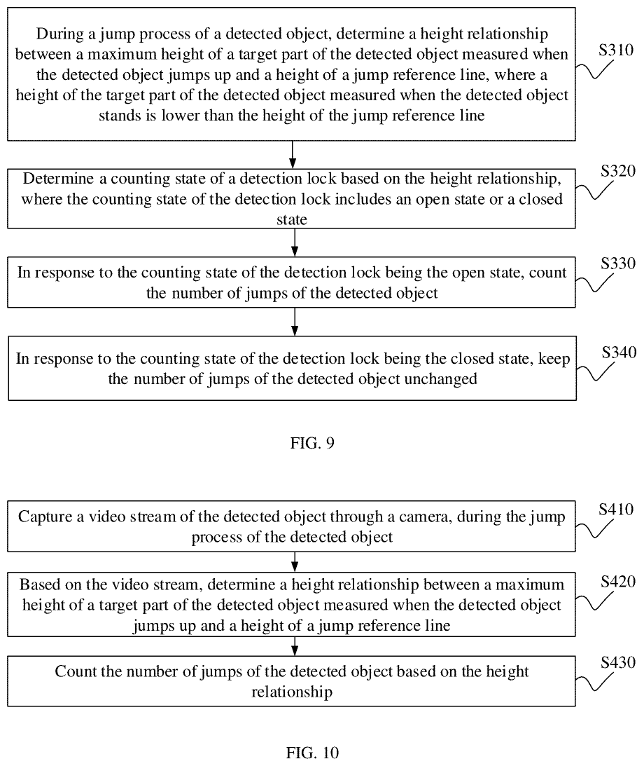

Referring to FIG. 9, a flowchart of the jump counting method provided in an embodiment of the present disclosure is schematically illustrated. The method is implemented by the electronic device mentioned above, and the flow illustrated in FIG. 9 will be described in detail below. The jump counting method may specifically include steps as follows.

In S310, during a jump process of a detected object, a height relationship between a maximum height of a target part of the detected object measured when the detected object jumps up and a height of a jump reference line is determined, where a height of the target part of the detected object measured when the detected object stands is lower than the height of the jump reference line.

For the detailed description of S310, reference may be made to the previous detailed description of S110, which is not repeated here.

In S320, a counting state of a detection lock is determined based on the height relationship, where the counting state of the detection lock includes an open state or a closed state.

In some implementations, during the jump process of the detected object, in determining the number of jumps of the detected object based on the height relationship between the maximum height of the target part of the detected object measured when the detected object jumps up and the height of the jump reference line, one jump of the detected object would be repeatedly counted, which causes low accuracy of counting the number of jumps of the detected object. In consideration of this, in the embodiment, in addition to the height relationship, the detection lock may be further introduced for determining the way of counting the number of jumps of the detected object, so as to improve the efficiency and accuracy of counting the number of jumps.

In the embodiment, the electronic device may determine a counting state of the detection lock based on the height relationship, where the counting state of the detection lock includes an open state or a closed state. In an alternative implementation, if the electronic device determines, based on the height relationship, that a current maximum height is changed from being equal to the height of the jump reference line to being higher than the height of the jump reference line, the counting state of the detection lock is adjusted from the open state to the closed state. If the electronic device determines, based on the height relationship, that the current maximum height is changed from being equal to the height of the jump reference line to being lower than the height of the jump reference line, the counting state of the detection lock is adjusted from the closed state to the open state.

That is, during a rising stage of each jump process of the detected object, if the current maximum height of the target part of the detected object is changed from being lower than the height of the jump reference line to being equal to the height of the jump reference line, the corresponding counting state of the detection lock is the open state; and if the height relationship is further changed to one that the current maximum height is higher than the height of the jump reference line, the counting state of the detection lock is correspondingly adjusted from the open state to the closed state. During a descending stage of the jump process of the detected object, if the current maximum height of the target part of the detected object is changed from being higher than the height of the jump reference line to being equal to the height of the jump reference line, the corresponding counting state of the detection lock is the closed state; and if the height relationship is further changed to one that the current maximum height is lower than the height of the jump reference line, the counting state of the detection lock is correspondingly adjusted from the closed state to the open state.

In S330, in response to the counting state of the detection lock being the open state, the number of jumps of the detected object is counted.

In the embodiment, when the counting state of the detection lock is the open state, the electronic device may count the number of jumps of the detected object. In an alternative implementation, the electronic device may determine the way of counting the number of jumps of the detected object, based on the height relationship between the maximum height of the target part of the detected object measured when the detected object jumps up and the height of the jump reference line, in combination with the counting state of the detection lock. Exemplarily, if the counting state of the detection lock is the open state, the number of jumps of the detected object is counted; specifically, if the counting state of the detection lock is the open state and the maximum height of the target part of the detected object measured when the detected object jumps up is equal to the height of the jump reference line, the number of jumps of the detected object is increased.

In S340, in response to the counting state of the detection lock being the closed state, the number of jumps of the detected object is kept unchanged, that is, the number of jumps of the detected object is not increased.

In the embodiment, when the counting state of the detection lock is the closed state, the electronic device keeps the number of jumps of the detected object unchanged. Exemplarily, if the counting state of the detection lock is the closed state, the number of jumps of the detected object is kept unchanged.

It is understandable that, only when the detection lock is in the open state, the electronic device counts the number of jumps of the detected object; and if the detection lock is in the closed state, the electronic device does not increase the number of jumps of the detected object. Thus, the accuracy of counting the number of jumps is improved.

Compared with the jump counting method illustrated in FIG. 1, the jump counting method provided in the embodiment of the present disclosure may further determine the counting state of the detection lock based on the height relationship between the maximum height of the target part of the detected object measured when the detected object jumps up and the height of the jump reference line, where the counting state of the detection lock includes the open state or the closed state. If the counting state of the detection lock is the open state, the number of jumps of the detected object is counted; and if the counting state of the detection lock is the closed state, the number of jumps of the detected object is kept unchanged. As such, the way of counting the number of jumps of the detected object can be effectively determined, the efficiency and accuracy of counting the number of jumps are improved, and the user's experience is further improved.

Referring to FIG. 10, a flowchart of the jump counting method provided in an embodiment of the present disclosure is schematically illustrated. The method is implemented by the electronic device mentioned above, and the flow illustrated in FIG. 10 will be described in detail below. The jump counting method may specifically include steps as follows.

In S410, during a jump process of a detected object, a video stream of the detected object is captured by the camera.

In the embodiment, the electronic device may capture, during the jump process of the detected object, a video stream of the detected object through the camera. The video stream is composed of one or more video frames, and each video frame may include the whole or part of the detected object, which is not limited here.

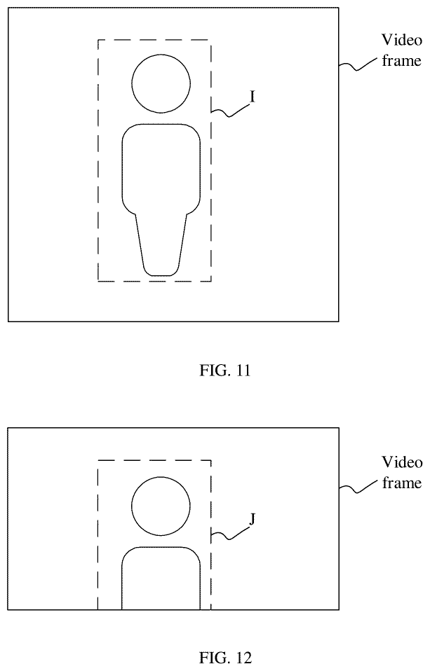

Exemplarily, referring to FIG. 11, a schematic diagram of a video frame provided in an embodiment of the present disclosure is illustrated. The video frame includes the whole of the detected object, as indicated by “I” in FIG. 11. Exemplarily, referring to FIG. 12, a schematic diagram of a video frame provided in an embodiment of the present disclosure is illustrated. The video frame includes part of the detected object, as indicated by “J” in FIG. 12.

In S420, based on the video stream, a height relationship between a maximum height of a target part of the detected object measured when the detected object jumps up and a height of a jump reference line is determined.

In the embodiment, after obtaining the video stream, the electronic device may determine, based on the video stream, the height relationship between the maximum height of the target part of the detected object measured when the detected object jumps up and the height of the jump reference line. The electronic device may detect, in each of the video frames included in the video stream, the maximum height of the target part of the detected object measured when the detected object jumps up and the height of the jump reference line, and then determine the height relationship between the maximum height of the target part of the detected object measured when the detected object jumps up and the height of the jump reference line. Exemplarily, the maximum height is higher than the height of the jump reference line, the maximum height is equal to the height of the jump reference line, or the maximum height is lower than the height of the jump reference line.

In an implementation, if the maximum height of the target part of the detected object measured when the detected object jumps up is higher than the height of the jump reference line in at least one of the video frames included in the video stream, it is determined that the maximum height is higher than the height of the jump reference line. If the maximum height of the target part of the detected object measured when the detected object jumps up is not higher than or not equal to the height of the jump reference line in each of the video frames included in the video stream, it is determined that the maximum height is lower than the height of the jump reference line. If the maximum height of the target part of the detected object measured when the detected object jumps up is not higher than the height of the jump reference line but is equal to the height of the jump reference line in at least one of the video frames included in the video stream, it is determined that the maximum height is equal to the height of the jump reference line.

In S430, the number of jumps of the detected object is counted based on the height relationship.

In the embodiment, after determining the height relationship, the electronic device may count the number of jumps of the detected object based on the height relationship.

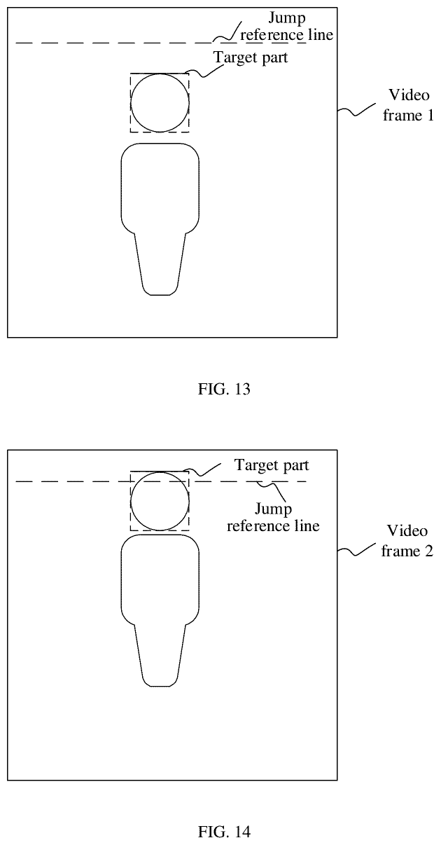

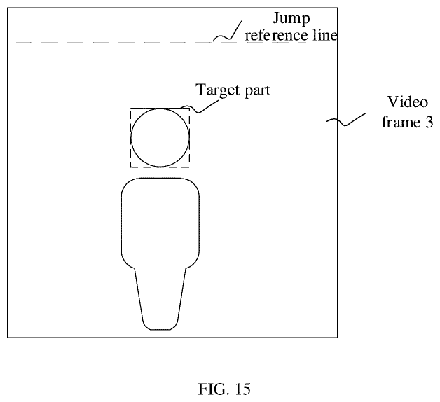

Exemplarily, referring to FIG. 13, FIG. 14 and FIG. 15, the electronic device may capture a video stream of the jump process of the detected object through the camera, where the video stream includes video frame 1 (as illustrated in FIG. 13), video frame 2 (as illustrated in FIG. 14) and video frame 3 (as illustrated in FIG. 15). The electronic device may perform the face detection on the detected object in each video frame, to obtain a face detection frame. Further, the electronic device may take an upper edge of the face detection frame as the target part of the detected object. The electronic device may determine, based on the video stream, the height relationship between the maximum height of the target part of the detected object measured when the detected object jumps up and the height of the jump reference line. The jump reference line may be a virtual line, and the electronic device may draw the virtual line in each of the video frames included in the video stream, as illustrated by the jump reference line in FIGS. 13, 14 and 15. As illustrated, the maximum height of the target part of the detected object is higher than the height of the jump reference line in video frame 2, then for this jump, the number of jumps of the detected object may be increased. The counting of the number of jumps of the detected object performed by the electronic device may end when the maximum height of the target part of the detected object is detected to be lower than the jump reference line (as illustrated in FIG. 15).

Compared with the jump counting method illustrated in FIG. 1, the jump counting method provided in the embodiment of the present disclosure further captures the video stream of the detected object through the camera, during the jump process of the detected object. The height relationship between the maximum height of the target part of the detected object measured when the detected object jumps up and the height of the jump reference line is determined based on the video stream. In this way, the height relationship is determined based on the video stream captured by the camera. It improves the efficiency and accuracy of the determination of the height relationship, thus improving the efficiency and accuracy of counting the number of jumps and improving the user's experience.

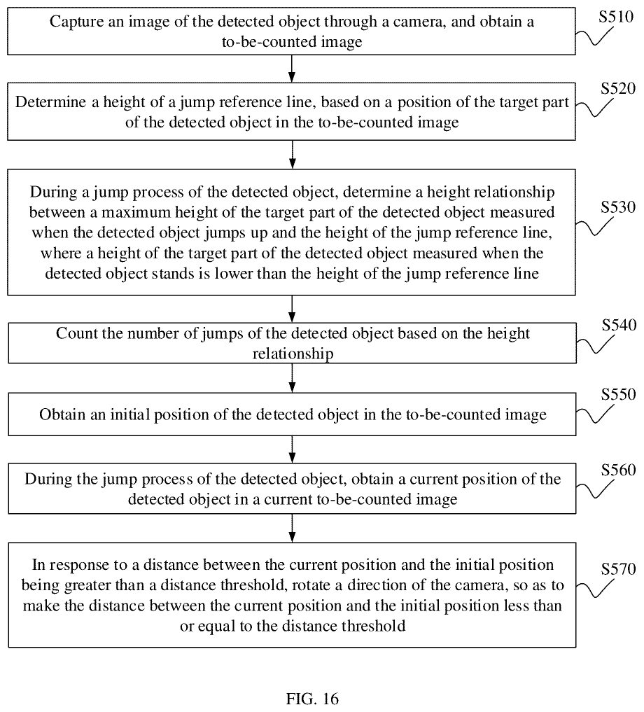

Referring to FIG. 16, a flowchart of the jump counting method provided in an embodiment of the present disclosure is schematically illustrated. The method is implemented by the electronic device mentioned above, and the flow illustrated in FIG. 16 will be described in detail below. The jump counting method may specifically include steps as follows.

In S510, an image of the detected object is captured through a camera, and a to-be-counted image is obtained.

In some implementations, the electronic device may capture an image of the detected image through the camera before the detected object jumps, and obtain a to-be-counted image. The to-be-counted image may include the target part of the detected object, or the whole or part of the detected object.

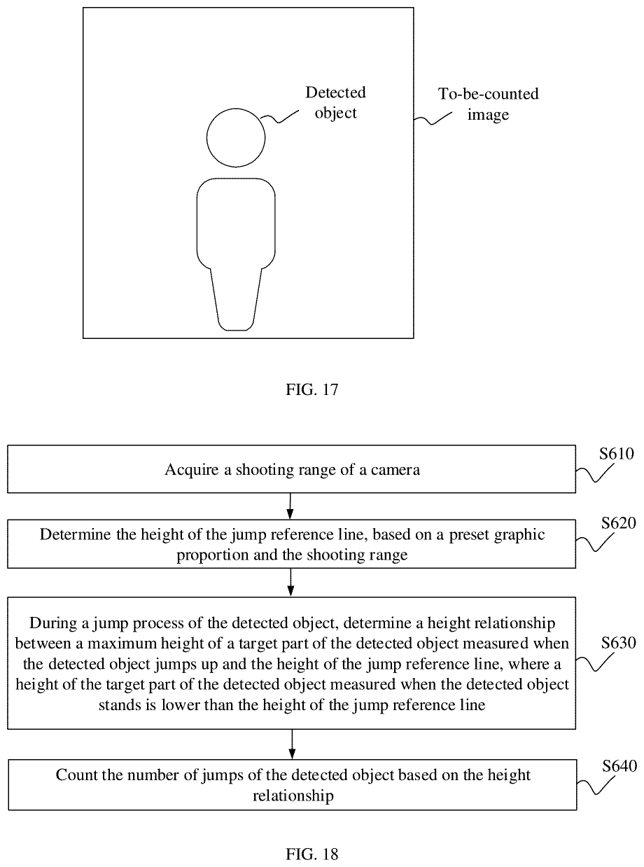

Exemplarily, referring to FIG. 17, a schematic diagram of a to-be-counted image provided in an embodiment of the present disclosure is shown, where the to-be- counted image includes the whole of the detected object.

In S520, a height of a jump reference line is determined based on a position of the target part of the detected object in the to-be-counted image.

In some implementations, after obtaining the to-be-counted image, the electronic device may determine the height of the jump reference line based on the position of the target part of the detected object in the to-be-counted image. In an alternative implementation, a preset relationship, such as a proportional relationship or a height difference relationship, between the height of the target part of the detected object and the height of the jump reference line in the to-be-counted image may be preset in the electronic device. Further, after determining the height of the target part of the detected object in the to-be-counted image, the electronic device may determine, based on the preset relationship, the height of the jump reference line in the to-be-counted image, so that the electronic device may count the number of jumps of the detected object in the to-be-counted images captured during subsequent jumps.

In some implementations, the to-be-counted image may include a plurality of detected objects. In an alternative implementation, the electronic device may determine, based on the target parts of the individual detected objects in the to-be-counted image, the jump reference lines respectively corresponding to the individual detected objects. Based on the jump reference lines respectively corresponding to the individual detected objects, the electronic device may further determine a highest one of these jump reference lines as the jump reference line for the plurality of the detected objects, or may also determine a lowest one of these jump reference lines as the jump reference line for the plurality of detected objects, or may also determine a jump reference line whose height is equal to for example the mode, median or average of the heights of these jump reference lines, as the jump reference line for the plurality of detected objects, which is not limited here.

In S530, during a jump process of the detected object, a height relationship between a maximum height of the target part of the detected object measured when the detected object jumps up and the height of the jump reference line is determined, where a height of the target part of the detected object measured when the detected object stands is lower than the height of the jump reference line.

In S540, the number of jumps of the detected object is counted based on the height relationship.

For the detailed description of S530-S540, reference may be made to the previous detailed description of S110-S120, which is not repeated here.

In S550, an initial position of the detected object in the to-be-counted image is obtained.

In some implementations, after obtaining the to-be-counted image, the electronic device may detect the position of the detected object in the to-be-counted image based on a preset position detection model (such as Cascade algorithm, HOG algorithm, DPM algorithm, Haar algorithm, and SVM algorithm), and obtain the initial position of the detected object in the to-be-counted image.

The electronic device may determine a center-of-gravity position of the detected object in the to-be-counted image as the initial position of the detected object, or may also determine a center position of the target part of the detected object in the to-be-counted image as the initial position of the detected object, which is not limited here.

In S560, during the jump process of the detected object, a current position of the detected object in a current to-be-counted image is obtained.

In some implementations, the electronic device may obtain, when the detected object jumps in a current to-be-counted image, the current position of the detected object in the to-be-counted image. In an alternative implementation, the electronic device may detect, when the detected object jumps in a current to-be-counted image, the position of the detected object in the current to-be-counted image based on a preset position detection model (such as Cascade algorithm, HOG algorithm, DPM algorithm, Haar algorithm, and SVM algorithm), and obtain the current position of the detected object in this to-be-counted image.

The current position of the detected object has the same attribute as the initial position of the detected object, and the current position of the detected object may be the center-of-gravity position of the detected object in the to-be-counted image, or the center position of the target part of the detected object in the to-be-counted image, which is not limited here. Exemplarily, the initial position of the detected object is the center-of-gravity position of the detected object, and the current position of the corresponding detected object is also the center-of-gravity position of the detected object.

In S570, if a distance between the current position and the initial position is greater than a distance threshold, a direction of the camera is rotated, so that the distance between the current position and the initial position is less than or equal to the distance threshold.

In some implementations, after determining the current position and the initial position of the detected object, the electronic device may calculate a distance between the current position and the initial position in the to-be-counted images. If the distance between the current position and the initial position is greater than the distance threshold, the direction of the camera is rotated, so that the distance between the current position in the to-be-counted image captured by the camera and the initial position is less than or equal to the distance threshold.

The distance threshold may be preset in the electronic device, or may also be obtained by the electronic device from an associated cloud or electronic device. The way of rotating the direction of the camera may include translating the camera, adjusting an angle of the camera, or adjusting a focal length of the camera, which is not limited here.

It is understandable that, the position would be moved during the jump process of the detected object. In consideration of this, in order to improve the accuracy of counting the number of jumps, in the embodiment, it is ensured that the distance between the initial position of the detected object in the to-be-counted image and the current position in the to-be-counted image during the jump process is less than or equal to the distance threshold, which improves the accuracy and efficiency of counting the number of jumps. In addition, by automatically adjusting the direction of the camera, the user's cooperation is avoided and the user's experience is improved.

Compared with the jump counting method illustrated in FIG. 1, the jump counting method provided in the embodiment of the present disclosure further captures an image of the detected object through the camera, and the to-be-counted image is obtained. The height of the jump reference line is determined based on the position of the target part of the detected object in a to-be-counted image. The initial position of the detected object in the to-be-counted image is acquired. During the jump process of the detected object, the current position of the detected object in a current to-be-counted image is obtained. If the distance between the current position and the initial position is greater than the distance threshold, the direction of the camera is rotated to make the distance between the current position and the initial position less than or equal to the distance threshold. In this way, the height of the jump reference line is flexibly determined based on the maximum height of the target part of the detected object, so that the applicability of the jump counting method is improved while the efficiency and accuracy of counting the number of jumps counting are improved. In addition, the electronic device may ensure the accuracy of counting the number of jumps by automatically adjusting the position of the camera, which improves the user's experience.

Referring to FIG. 18, a flowchart of the jump counting method provided in an embodiment of the present disclosure is schematically illustrated. The method is implemented by the electronic device mentioned above, and the flow illustrated in FIG. 18 will be described in detail below. The jump counting method may specifically include steps as follows.

In S610, a shooting range of a camera is acquired.

In some implementations, in order to reduce power consumption of the electronic device in counting the number of jumps, the electronic device may determine, based on the shooting range of the camera, a fixed jump reference line relative to the shooting range of the camera. The electronic device may acquire the shooting range of the camera, and further count the number of jumps of the detected object based on the shooting range.

In S620, a height of a jump reference line is determined, based on a preset graphic proportion and the shooting range.

In the embodiment, after obtaining the shooting range of the camera, the electronic device may determine the height of the jump reference line based on the preset graphic proportion and the shooting range. In an alternative implementation, the preset graphic proportion may be preset in the electronic device, or may be obtained by the electronic device from an associated cloud or electronic device, which is not limited here.

The preset graphic proportion may include a ratio of the height of the jump reference line to a height of a display picture corresponding to the shooting range, or may also include a distance between the height of the jump reference line and a top of the display picture corresponding to the shooting range, which is not limited here.

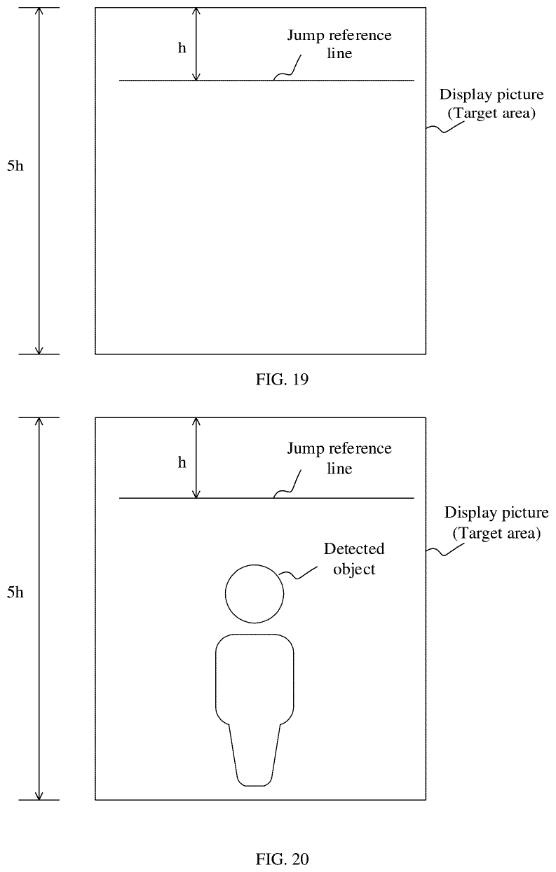

Exemplarily, referring to FIG. 19, a schematic diagram of a jump reference line provided in an embodiment of the present disclosure is illustrated. The preset graphic proportion includes a ratio of the distance between the height of the jump reference line and the top of the display picture corresponding to the shooting range to the height of the display picture, such as a ratio of 20%. The display picture corresponding to the shooting range, as illustrated in FIG. 19, has a height of 5 h. The ratio of the distance between the height of the jump reference line and the top of the display picture to the height of the display picture is 20%. As illustrated in FIG. 19, the distance between the height of the jump reference line and the top of the display picture is h.

In some implementations, after acquiring the shooting range of the camera, the electronic device may present the shooting range on an associated screen. Further, after determining the height of the jump reference line based on the preset graphic proportion and the shooting range, the electronic device may further draw the jump reference line on the display picture corresponding to the shooting range, and display the jump reference line on the associated screen.

In an implementation, the electronic device may display the jump reference line in a target area, so that the detected object may jump based on the jump reference line. The target area may be construed as an area facing the detected object. The target area may further be construed as the display picture corresponding to the shooting range, or may also be construed as an area at the height of the jump reference line in the display picture corresponding to the shooting range, which is not limited here.

Exemplarily, referring to FIG. 19 again, a schematic diagram of displaying a jump reference line provided in an embodiment of the present disclosure is illustrated. The electronic device displays the jump reference line in the target area, so that the detected object jumps based on the jump reference line. The target area is an area to which the detected object faces, which may also be construed as the display picture corresponding to the shooting range. Further, referring to FIG. 20, a display picture provided in an embodiment of the present disclosure is illustrated. The detected object may jump based on the jump reference line.

As an implementation, the electronic device may further display a standing area of the detected object when presenting the shooting range, or may also display the standing area of the detected object when displaying the jump reference line in the target area, so that the user may stand and jump based on the standing area. This improves the efficiency and accuracy of counting the number of jumps, and improves the user's experience.

In S630, during a jump process of the detected object, a height relationship between a maximum height of a target part of the detected object measured when the detected object jumps up and the height of the jump reference line is determined, where a height of the target part of the detected object measured when the detected object stands is lower than the height of the jump reference line.

For the detailed description of S630, reference may be made to the previous detailed description of S110, which is not repeated here.

In S640, the number of jumps of the detected object is counted based on the height relationship.

In some implementations, the electronic device may count the number of jumps of the detected object within a target time period. A time duration of the target time period may be equal to, longer than or shorter than the first jump counting time. The target time period may be 30 seconds, 1 minute, 2 minutes, etc., which is not limited here.

Further, the electronic device may accumulate the number of jumps of the detected object within the target time period. In an alternative implementation, the electronic device may generate first prompt information based on the number of jumps of the detected object. The first prompt information may include the counted number of jumps of the detected object. The first prompt information may also include the time duration during which the electronic device counts the number of jumps of the detected object. The first prompt information may be voice prompt information, text prompt information, etc., which is not limited here.

Exemplarily, the electronic device may display the first prompt information in the target area. A position where the first prompt information is displayed in the target area may be an upper left corner of the target area, or a lower right corner of the target area, which is not limited here.

In some implementations, during the jumps process of the detected object, the electronic device may determine a changing situation of the number of jumps of the detected object within a counting time, and generate second prompt information based on the changing situation. The changing situation of the number of jumps may include that the number of jumps is kept unchanged or is increased. Here, the counting time may be equal to or less than the target time period, which is not limited here.

As an implementation, if the electronic device determines, based on the changing situation, that the number of jumps of the detected object is kept unchanged, the second prompt information is generated. The second prompt information may be configured to prompt that the jump is failed, The second prompt information may be voice prompt information, text prompt information, etc., which is not limited here.

Exemplarily, the electronic device may display the second prompt information in the target area. A position where the second prompt information is displayed in the target area may be the upper left corner, or the lower right corner of the target area, which is not limited here.

The second prompt information may further include the distance between the height of the target part measured when the detected object stands and the height of the jump reference line, so that the user may jump based on the prompt information, which improves the user's experience.

In some implementations, during the jump process of the detected object, the electronic device may acquire the jump state of the detected object, and generate interactive information based on the jump state. The interactive information may include voice information, animation information, text information, etc., which is not limited here. Exemplarily, the interactive information is animation information, and the electronic device may display the interactive information in the target area.

The jump state of the detected object may include the maximum height of the target part and a jump duration of the detected object during the jump process of the detected object, and the like, which is not limited here. In an alternative implementation, the interactive information generated by the electronic device based on the jump state may include interactive information of praise and interactive information of refueling that are generated based on the jump state, so that the user's experience is improved.

Exemplarily, if it is determined that the maximum height is higher than an encouraging height value, the interactive information of praise, such as voice information “You are great” may be generated. If it is determined that the jump duration of the detected object is equal to an encouraging duration, the interactive information of refueling, such as voice information “Hold on”, may be generated.

Compared with the jump counting method illustrated in FIG. 1, in the jump counting method provided in the embodiment of the present disclosure, the shooting range of the camera may be acquired, and the height of the jump reference line is determined based on the preset graphic proportion and the shooting range. As such, the height of the jump reference line is determined based on the shooting range of the camera and the preset graphic proportion. Then, the number of jumps is counted based on the height, which improves the efficiency and accuracy of counting the number of jumps and improves the user's experience.

Referring to FIG. 21, a block diagram of a jump counting apparatus provided in an embodiment of the present disclosure is illustrated. The jump counting apparatus 200 may be applied to the electronic device mentioned above, and the flow illustrated in FIG. 21 will be described in detail below. The jump counting apparatus 200 includes a height relationship determining module 210 and a jump counting module 220.

The height relationship determining module 210 is configured to determine, during a jump process of a detected object, a height relationship between a maximum height of a target part of the detected object measured when the detected object jumps up and a height of a jump reference line, where a height of the target part of the detected object measured when the detected object stands is lower than the height of the jump reference line.

The jump counting module 220 is configured to count the number of jumps of the detected object, based on the height relationship.

Further, the jump counting module 220 may include a number-increasing unit or a number-keeping unit.

The number-increasing unit is configured to increase the number of jumps of the detected object when it is determined, based on the height relationship, that the maximum height is higher than or equal to the height of the jump reference line.

The number-keeping unit is configured to keep the number of jumps of the detected object unchanged when it is determined, based on the height relationship, that the maximum height is lower than the height of the jump reference line.

Further, the number-increasing unit may include a height difference determining unit, a first counting value determining unit and a first number-increasing unit.

The height difference determining unit is configured to determine a height difference between the maximum height and the height of the jump reference line, when it is determined, based on the height relationship, that the maximum height is higher than or equal to the height of the jump reference line.

The first counting value determining unit is configured to determine the first counting value based on the height difference, where the first counting value is greater than or equal to 1.

The first number-increasing unit is configured to increase the number of jumps of the detected object, based on the first counting value.

Further, the number-increasing unit may include a duration determining unit, a second counting value determining unit and a second number-increasing unit.

The duration determining unit is configured to determine a duration during which the maximum height is higher than or equal to the height of the jump reference line, when it is determined, based on the height relationship, that the maximum height is higher than or equal to the height of the jump reference line.

The second counting value determining unit is configured to determine a second counting value based on the duration, where the second counting value is greater than or equal to 1.

The second number-increasing unit is configured to increase the number of jumps of the detected object based on the second counting value.

Further, the number-increasing unit may include a hand rotation number determining unit, a third counting value determining unit and a third number-increasing unit.

The hand rotation number determining unit is configured to determine a hand rotation number of the detected object, when it is determined, based on the height relationship, that the maximum height is higher than or equal to the height of the jump reference line.

The third counting value determining unit is configured to determine a third counting value based on the hand rotation number, where the third counting value is greater than or equal to 1.

The third number-increasing unit is configured to increase the number of jumps of the detected object based on the third counting value.

Further, the jump counting module 220 may further include a counting state determining unit, a number-increasing unit and a number-keeping unit.

The counting state determining unit is configured to determine, based on the height relationship, a counting state of the detection lock, where the counting state of the detection lock includes an open state or a closed state.

The number-increasing unit is configured to count the number of jumps of the detected object, in response to the counting state of the detection lock being the open state.

The counting invariant unit is configured to keep the number of jumps of the detected object, in response to the counting state of the detection lock being the closed state.

Further, the counting state determining unit may include an open-to-closed adjusting unit or a closed-to-open adjusting unit.

The open-to-closed adjusting unit is configured to adjust the counting state of the detection lock from the open state to the closed state, in response to determining, based on the height relationship, that the maximum height is changed from being equal to the height of the jump reference line to being higher than the height of the jump reference line.

The closed-to-open adjusting unit is configured to adjust the counting state of the detection lock from the closed state to the open state, in response to determining, based on the height relationship, that the maximum height is changed from being equal to the jump reference line to being lower than the height of the jump reference line.

Further, the height relationship determining module 210 may include a face acquiring unit, a target part determining unit and a first height relationship determining unit.

The face acquiring unit is configured to, during the jump process of the detected object, perform face detection on the detected object, to obtain a face of the detected object.

The target part determining unit is configured to determine an upper edge of the face as the target part of the detected object.

The first height relationship determining unit is configured to determine the height relationship between the maximum height of the target part of the detected object measured when the detected object jumps up and the height of the jump reference line.

Further, the face acquiring unit may include a target image capturing unit and a model-based face detection unit.

The target image capturing unit is configured to capture a target image including the detected object through a camera, during the jump process of the detected object.

The model-based face detection unit is configured to input the target image into a face detection model and obtain the face of the detected object in the target image output by the face detection model.

Further, the height relationship determining module 210 may include a video stream capturing unit and a second height relationship determining unit.

The video stream capturing unit is configured to capture a video stream of the detected object through a camera, during the jump process of the detected object.

The second height relationship determining unit is configured to determine, based on the video stream, the height relationship between the maximum height of the target part of the detected object measured when the detected object jumps up and the height of the jump reference line.

Further, during the jump process of the detected object and before determining the height relationship between the maximum height of the target part of the detected object measured when the detected object jumps up and the height of the jump reference line, the jump counting apparatus 200 may further include a to-be- counted image obtaining module and a first jump-reference-line's height determining module.

The to-be-counted image obtaining module is configured to capture an image of the detected object through a camera, to obtain a to-be-counted image.

The first jump-reference-line's height determining module is configured to determine the height of the jump reference line based on a position of the target part of the detected object in the to-be-counted image.

Further, the jump counting apparatus 200 may further include an initial position acquiring module, a current position determining module and a camera rotation module.

The initial position acquiring module is configured to acquire an initial position of the detected object in the to-be-counted image.

The current position determining module is configured to acquire, during the jump process of the detected object, a current position of the detected object in a current to-be-counted image.

The camera rotation module is configured to rotate a direction of the camera when a distance between the current position and the initial position is greater than a distance threshold, so as to make the distance between the current position and the initial position less than or equal to the distance threshold.

Further, during the jump process of the detected object and before determining the height relationship between the maximum height of the target part of the detected object measured when the detected object jumps up and the height of the jump reference line, the jump counting apparatus 200 may further include a shooting range acquiring module and a second jump-reference-line's height determining module.

The shooting range acquiring module is configured to acquire a shooting range of the camera.

The second jump-reference-line's height determining module is configured to determine the height of the jump reference line, based on a preset graphic proportion and the shooting range.

Further, during the jump process of the detected object and before determining the height relationship between the maximum height of the target part of the detected object measured when the detected object jumps up and the height of the jump reference line, the jump counting apparatus 200 may further include a jump reference line display module.

The jump reference line display module is configured to display the jump reference line in a target area, so that the detected object jumps based on the jump reference line, where the target area is the area to which the detected object faces.

Further, the jump counting apparatus 200 may further include a first prompt information generating module and a first prompt information display module.

The first prompt information generating module is configured to generate first prompt information based on the number of jumps of the detected object, where the first prompt information includes the number of jumps of the detected object.

The first prompt information display module is configured to display the first prompt information in the target area.

Further, the jump counting apparatus 200 may further include a number change determining module, a second prompt information generating module and a second prompt information display module.

The number change determining module is configured to determine a changing situation of the number of jumps of the detected object within a counting time, during the jump process of the detected object.

The second prompt information generating module is configured to generate second prompt information, in response to determining, based on the changing situation, that the number of jumps of the detected object is kept unchanged, where the second prompt information is configured to prompt failure of that the jump.

The second prompt information display module is configured to display the second prompt information in the target area.

Further, the jump counting apparatus 200 may further include a jump state acquiring module, an interactive information generating module and an interactive information display module.

The jump state acquiring module is configured to acquire a jump state of the detected object during the jump process of the detected object.

The interactive information generating module is configured to generate interactive information based on the jump state.

The interactive information display module is configured to display the interactive information in the target area.

It may be clearly appreciated by those skilled in the art that, for the convenience and conciseness of description, specific working processes of the apparatus and modules described above may refer to the corresponding processes in the method embodiments mentioned above, which are not be repeated here.

In several embodiments provided in the present disclosure, coupling between modules may be electrical, mechanical or in other forms.

In addition, the individual functional modules in the embodiments of the present disclosure may be integrated into one processing module, or each of the modules may exist physically alone, or two or more modules may be integrated into one module. The integrated modules above may be realized in the form of hardware or software functional modules.

Referring to FIG. 22, a structural block diagram of an electronic device 100 provided in an embodiment of the present disclosure is illustrated. The electronic device 100 may be a smart phone, a tablet computer, an e-book and other electronic devices capable of running application programs. The electronic device 100 in the present disclosure may include one or more of the following components: a processor 110, a memory 120, and one or more application programs, where the one or more application programs may be stored in the memory 120 and configured to be executed by one or more processors 110 to cause the method as described in the method embodiment mentioned before to be implemented.

The processor 110 may include one or more processing cores. The processor 110 connects individual parts of the whole electronic device 100 using various interfaces and lines, and executes various functions and processes data of the electronic device 100 by running or executing instructions, programs, code sets or instruction sets stored in the memory 120 and calling data stored in the memory 120. Alternatively, the processor 110 may be implemented in at least one hardware form of Digital Signal Processor (DSP), Field-Programmable Gate Array (FPGA) and Programmable Logic Array (PLA). The processor 110 may be integrated with one or more combinations of Central Processor Unit (CPU), Graphics Processor Unit (GPU) and a modem. Among them, CPU mainly deals with operating system, user interface and application programs. GPU is configured to render and draw to-be-displayed content. The modem is configured to handle wireless communication. It is understandable that the modem above may further be realized by a communication chip without being integrated into the processor 110.

The memory 120 may include a Random Access Memory (RAM), and may also include a Read-Only Memory (ROM). The memory 120 may be configured to store instructions, programs, codes, code sets or instruction sets. The memory 120 may include a program storage area and a data storage area, where the program storage area may store instructions for implementing an operating system, instructions for implementing at least one function (such as a touch function, a sound playing function, and an image display function), and instructions for implementing various method embodiments described here. The data storage area may further store data (such as phone book, audio and video data, chat record data) created by the electronic device 100 in use.

Referring to FIG. 23, a structural block diagram of a computer-readable storage medium provided in an embodiment of the present disclosure is illustrated. The computer-readable medium 300 stores program codes therein, and the program codes may be called by the processor to execute the method described in the method embodiments above.

The computer-readable storage medium 300 may be an electronic memory such as flash memory, Electrically Erasable Programmable Read Only Memory (EEPROM), EPROM, hard disk or ROM. Alternatively, the computer-readable storage medium 300 includes a non-volatile/transitory computer-readable storage medium. The computer-readable storage medium 300 has a storage space for program codes 310 for executing any method step in the method above. These program codes may be read from or written into one or more computer program products. The program codes 310 may be compressed in an appropriate form.

To sum up, in the jump counting method and apparatus, the electronic device and the storage medium provided in the embodiments of the present disclosure, during a jump process of a detected object, the height relationship between the maximum height of a target part of the detected object measured when the detected object jumps up and the height of the jump reference line is determined, where the height of the target part of the detected object measured when the detected object stands is lower than the height of the jump reference line. The number of jumps of the detected object is counted based on the height relationship. As such, during the jump process of the detected object, the number of jumps of the detected object is automatically performed based on the height relationship between the maximum height of the target part of the detected object and the jump reference line, which improves the efficiency of the number of jumps.

Finally, it is notable that the embodiments above are only used to illustrate the technical schemes of the present disclosure, rather than limiting it. Although the present disclosure are described in detail with reference to the foregoing embodiments, it is understandable for those skilled in the art that the technical schemes described in the foregoing embodiments may still be modified, or some technical features described in the foregoing embodiments may be replaced by equivalents. These modifications or replacements do not drive the essence of the corresponding technical schemes to depart away from the spirit and scope of the technical schemes of various embodiments of the present disclosure.

What is claimed is:

1. A jump counting method, comprising:
during a jump process of a detected object, determining a height relationship between a maximum height of a target part of the detected object measured when the detected object jumps up and a height of a jump reference line, wherein a height of the target part of the detected object measured when the detected object stands is lower than the height of the jump reference line; and
counting the number of jumps of the detected object, based on the height relationship.

2. The method of claim 1, wherein counting the number of jumps of the detected object based on the height relationship comprises:
in response to determining, based on the height relationship, that the maximum height is higher than or equal to the height of the jump reference line, increasing the number of jumps of the detected object; or
in response to determining, based on the height relationship, that the maximum height is lower than the height of the jump reference line, keeping the number of jumps of the detected object unchanged.

3. The method of claim 2, wherein increasing the number of jumps of the detected object in response to determining, based on the height relationship, that the maximum height is higher than or equal to the height of the jump reference line, comprises:
in response to determining, based on the height relationship, that the maximum height is higher than or equal to the height of the jump reference line, determining a height difference between the maximum height and the height of the jump reference line;
determining a first counting value based on the height difference, wherein the first counting value is greater than or equal to 1; and
increasing the number of jumps of the detected object based on the first counting value.

4. The method of claim 2, wherein increasing the number of jumps of the detected object in response to determining, based on the height relationship, that the maximum height is higher than or equal to the height of the jump reference line, comprises:
in response to determining, based on the height relationship, that the maximum height is higher than or equal to the height of the jump reference line, determining a duration during which the maximum height is higher than or equal to the height of the jump reference line;
determining a second counting value based on the duration, wherein the second counting value is greater than or equal to 1; and
increasing the number of jumps of the detected object based on the second counting value.

5. The method of claim 2, wherein increasing the number of jumps of the detected object in response to determining, based on the height relationship, that the maximum height is higher than or equal to the height of the jump reference line, comprises:
in response to determining, based on the height relationship, that the maximum height is higher than or equal to the height of the jump reference line, determining a hand rotation number;
determining a third counting value based on the hand rotation number, wherein the third counting value is greater than or equal to 1; and
increasing the number of jumps of the detected object based on the third counting value.

6. The method of claim 1, wherein counting the number of jumps of the detected object based on the height relationship comprises:
determining a counting state of a detection lock based on the height relationship, wherein the counting state of the detection lock comprises an open state or a closed state;
in response to the counting state of the detection lock being the open state, counting the number of jumps of the detected object; or
in response to the counting state of the detection lock being the closed state, keeping the number of jumps of the detected object unchanged.

7. The method of claim 6, wherein determining the counting state of the detection lock based on the height relationship comprises:
in response to determining, based on the height relationship, that a current maximum height is changed from being equal to the height of the jump reference line to being higher than the height of the jump reference line, adjusting the counting state of the detection lock from the open state to the closed state; or
in response to determining, based on the height relationship, that the current maximum height is changed from being equal to the height of the jump reference line to being lower than the height of the jump reference line, adjusting the counting state of the detection lock from the closed state to the open state.

8. The method of claim 1, wherein during the jump process of the detected object, determining the height relationship between the maximum height of the target part of the detected object measured when the detected object jumps up and the height of the jump reference line comprises:
during the jump process of the detected object, performing face detection on the detected object, and obtaining a face of the detected object from the face detection;
determining an upper edge of the face as the target part of the detected object; and
determining the height relationship between the maximum height of the target part of the detected object measured when the detected object jumps up and the height of the jump reference line.

9. The method of claim 8, wherein during the jump process of the detected object, performing face detection on the detected object and obtaining the face of the detected object from the face detection comprises:
capturing a target image comprising the detected object through a camera, during the jump process of the detected object; and
inputting the target image into a face detection model, and obtaining the face of the detected object in the target image output by the face detection model.

10. The method of claim 1, wherein during the jump process of the detected object, determining the height relationship between the maximum height of the target part of the detected object measured when the detected object jumps up and the height of the jump reference line comprises:
capturing a video stream of the detected object through a camera, during the jump process of the detected object; and
determining, based on the video stream, the height relationship between the maximum height of the target part of the detected object measured when the detected object jumps up and the height of the jump reference line.

11. The method of claim 1, wherein during the jump process of the detected object, and before determining the height relationship between the maximum height of the target part of the detected object measured when the detected object jumps up and the height of the jump reference line, the method further comprises:
capturing an image of the detected object through a camera, and obtaining a to-be- counted image through the capturing; and
determining the height of the jump reference line, based on a position of the target part of the detected object in the to-be-counted image.

12. The method of claim 11, wherein the method further comprises:
acquiring an initial position of the detected object in the to-be-counted image;
acquiring, during the jump process of the detected object, a current position of the detected object in a current to-be-counted image; and
in response to a distance between the current position and the initial position being greater than a distance threshold, rotating a direction of the camera, so as to make the distance between the current position and the initial position less than or equal to the distance threshold.

13. The method of claim 1, wherein during the jump process of the detected object, and before determining the height relationship between the maximum height of the target part of the detected object measured when the detected object jumps up and the height of the jump reference line, the method further comprises:
acquiring a shooting range of a camera;
determining the height of the jump reference line, based on a preset graphic proportion and the shooting range.

14. The method of claim 1, wherein during the jump process of the detected object, and before determining the height relationship between the maximum height of the target part of the detected object measured when the detected object jumps up and the height of the jump reference line, the method further comprises:
displaying the jump reference line in a target area, so that the detected object jumps based on the jump reference line, wherein the target area is an area to which the detected object faces.

15. The method of claim 14, wherein the method further comprises:
generating first prompt information based on the number of jumps of the detected object, wherein the first prompt information comprises the number of jumps of the detected object; and
displaying the first prompt information in the target area.

16. The method of claim 14, wherein the method further comprises:
determining a changing situation of the number of jumps of the detected object within a counting time, during the jump process of the detected object;
in response to determining, based on the changing situation, that the number of jumps of the detected object is kept unchanged, generating second prompt information, wherein the second prompt information is configured to prompt failure of the jump; and
displaying the second prompt information in the target area.

17. The method of claim 14, wherein the method further comprises:
acquiring a jump state of the detected object during the jump process of the detected object;
generating interactive information based on the jump state; and
displaying the interactive information in the target area.

18. An electronic device, comprising:
one or more processors;
a memory; and
one or more application programs, wherein the one or more application program s are stored in the memory, and configured to be executed by the one or more processors to:
during a jump process of a detected object, determine a height relationship between a maximum height of a target part of the detected object measured when the detected object jumps up and a height of a jump reference line, wherein a height of the target part of the detected object measured when the detected object stands is lower than the height of the jump reference line; and
count the number of jumps of the detected object, based on the height relationship.

19. The electronic device of claim 18, wherein the processor is further configured to:
in response to determining, based on the height relationship, that the maximum height is higher than or equal to the height of the jump reference line, increase the number of jumps of the detected object; or
in response to determining, based on the height relationship, that the maximum height is lower than the height of the jump reference line, keep the number of jumps of the detected object unchanged.

20. A non-transitory computer-readable storage medium storing program codes therein, wherein the program codes are invokable by a processor to:
during a jump process of a detected object, determine a height relationship between a maximum height of a target part of the detected object measured when the detected object jumps up and a height of a jump reference line, wherein a height of the target part of the detected object measured when the detected object stands is lower than the height of the jump reference line; and
in response to determining, based on the height relationship, that the maximum height is higher than or equal to the height of the jump reference line, increase the number of jumps of the detected object.
